(19) United States
(12) Patent Application Publication
(10) Pub. No.: US 2025/0025412 A1
(43) Pub. Date: Jan. 23, 2025

(54) Personal Lubricants With Wellness Additive Components

(71) Applicants: Sabrina Michelle Phillips, Roswell, GA (US); Justin Michael Czerniawski, Roswell, GA (US)

(72) Inventors: Sabrina Michelle Phillips, Roswell, GA (US); Justin Michael Czerniawski, Roswell, GA (US)

(21) Appl. No.: 18/768,175

(22) Filed: Jul. 10, 2024

Related U.S. Application Data
(63) Continuation of application No. 63/513,965, filed on Jul. 17, 2023.

(51) Int. Cl. A61K 9/00; A61K 45/06; A61K 47/34; A61K 47/44

(52) U.S. Cl. CPC A61K 9/0034; A61K 45/06; A61K 47/34; A61K 47/44

(57) ABSTRACT

A personal lubrication formula comprising a water, oil, or silicone-based lubricant with a wellness additive is disclosed. The formula uses the lubricant as a carrier for wellness additives such as amino acid complexes, vitamin C, vitamin D, vitamin B complexes, caffeine, creatine, and ashwagandha. The wellness additive is dissolved in the formula. The formula with wellness additives dissolved allows for the delivery of the wellness additives to the body during intimate contact with the sex organ mucous membranes. A method of using a personal lubrication formula for delivering wellness additives to a sex organ mucous membrane is also disclosed. When used during intimate contact, the lubricant comes into contact with sex organ mucous membranes. The personal lubricant acts as a carrier for wellness additives. These additives are then transferred to the users by absorption from contact with mucous membranes to provide health and wellness benefits to the user.

### FIELD OF THE INVENTION

The present application relates to personal lubricant formula, personal lubricant composition, health, wellness supplements, wellness additives, membranes absorption, methods for delivery of wellness supplements particularly the absorption of wellness supplements using a personal lubricant composition as a carrier of wellness supplements, absorbed through sex organ mucous membranes after use of the personal lubricant with wellness additives. The composition includes at least one wellness additive which provides a method of wellness additive absorption upon contact with sex organ mucous membranes.

### BACKGROUND

Wellness is quickly becoming a consumer focus. It is very common for research and health professionals to recommend daily supplements to help with wellness aspects such as energy, pain relief, focus, libido, and weight loss. Some examples of well-researched wellness supplements are amino acid complexes, vitamin C, vitamin D, vitamin B complexes, caffeine, creatine, and ashwagandha. These supplements are soluble and absorbed through mucous membranes in the body, primarily through ingestion in pill form. Since there are absorbing mucous membranes in the sex organs (oral, rectal, vaginal), the sex organ mucous membranes can also absorb these supplements (such as the examples mentioned above) if they are dissolved into a media as a wellness additive. The personal lubricant market is currently experiencing a shift to more natural and wellness-focused composition (organic ingredients, aloe additives, coconut oil). Personal lubricant compositions primarily come in 3 varieties, water-based, silicone-based, and oil-based. Wellness additives are soluble in these bases. As the focus on wellness increases in these two areas, there remains a need to combine sexual health and supplement health for a better consumer experience.

### SUMMARY

A system and accompanying method for providing a personal lubricant composition where at least one wellness additive is dissolved in the lubricant are disclosed. In particular, the system and methods take advantage of the discovery that personal lubricants can deliver dissolved wellness additives through sex mucous membranes during intimate contact such as intercourse or masturbation.

In certain embodiments, a personal lubrication composition would have a single wellness additive, such as caffeine, dissolved into the lubricant. This lubricant would come into contact with absorbing mucous membranes during sexual intercourse or masturbation. The dissolved caffeine in the lubricant would come into contact with the absorbing mucous membrane and cross into the body, delivering caffeine's health benefits to the users.

In one embodiment, a personal lubrication composition would have multiple wellness additives, such as vitamin D and vitamin C, dissolved into the lubricant. This lubricant would come into contact with absorbing mucous membranes during sexual intercourse or masturbation. The dissolved vitamins in the lubricant would come into contact with the absorbing mucous membrane and cross into the body, delivering the vitamins' health benefits to the users.

In another embodiment, the personal lubrication composition with the wellness additive could be in various forms such as, but not limited to, a liquid, gel, paste, bar, or balm.

In another embodiment, a method for delivering wellness supplements using personal lubricants as a carrier is disclosed. Wellness additives dissolve into either water, oil, or silicone-based personal lubricants as a method of delivery upon contact with sex mucous membranes during intercourse or masturbation. This method can be used for both personal and medical applications.

These and other features of the systems and methods for providing a personal lubricant composition where at least one wellness additive is dissolved in the lubricant are described in the following detailed description, drawings, and appended claims.

We claim:

1. A personal lubrication formula, comprising:
a water, silicone, or oil-based personal lubricant formula; and
a wellness additive.

2. The personal lubrication formula of claim 1, wherein the formula includes one or more wellness additives.

3. The personal lubrication formula of claim 1, wherein the water, silicone, or oil-based personal lubricant formula is a carrier of a wellness additive.

4. The personal lubrication formula of claim 1, wherein the formula includes an emulsifier to dissolve the wellness additive.

5. The personal lubrication formula of claim 1, wherein the form can be but is not limited to a liquid, gel, paste, bar, or balm.

6. The personal lubrication formula of claim 2, further comprises a wellness additive used to provide health benefits such as but not limited to pain relief, mood enhancement, immune support, or energy enhancement.

7. A method, comprising:
The use of a personal lubrication formula as a carrier for the delivery of wellness additive to a sex organ mucous membrane for absorption.

8. The method of claim 7, wherein one or more wellness additives are delivered to the mucous membrane through contact of the lubrication formula with a mucous membrane.

9. The method of claim 7, wherein the method is used to provide health benefits such as but not limited to pain relief, mood enhancement, immune support, or energy enhancement.
